(19) United States
(12) Patent Application Publication
(10) Pub. No.: US 2023/0371789 A1
(43) Pub. Date: Nov. 23, 2023

(54) ENDOSCOPE WITH SELF-ENCASED SELF-ILLUMINATING WIDE-ANGLE PANORAMIC IMAGING MODULE

(71) Applicant: Nitesh Ratnakar, Wheeling, WV (US)

(72) Inventor: Nitesh Ratnakar, Wheeling, WV (US)

(21) Appl. No.: 15/917,567

(22) Filed: Mar. 9, 2018

Related U.S. Application Data
(60) Provisional application No. 62/468,963, filed on Mar. 9, 2017.

(51) Int. Cl. A61B 1/00; A61B 1/05; A61B 1/06

(52) U.S. Cl. CPC A61B 1/00096; A61B 1/05; A61B 1/0676

(57) ABSTRACT

The present disclosure relates to a single-shaft endoscope having a single shaft. The endoscope comprises a housing at a distal end of the single shaft. The housing is configured to be self-encased, optically clear, and self-illuminating. The endoscope further comprises a camera deployed inside the housing. The camera is configured to provide a wide-angle panoramic view.

### CROSS-REFERENCE TO RELATED APPLICATION

This application claims the benefit under 35 U.S.C. § 119(e) of Provisional Patent Application No. 62/468,963, filed Mar. 9, 2017, the entire disclosure of which is hereby incorporated by reference.

### RELATED ART

### 1. Technical Field

The present disclosure generally relates to an endoscope with self-encased and self-illuminating wide-angle panoramic imaging module at its distal end to capture images of a wide angle (which can be as wide as 360 degree or near 360 degree) about a lumen (such as a gastrointestinal lumen) where the distal end is interposed during an endoscopy session.

### 2. Description of the Related Art

A conventional single-shaft endoscope is usually equipped with one or more imaging modules at or adjacent to the distal end 103 of its single shaft 101 having a hollow channel 102 (extending from the proximal end 104 to the distal end 103). To capture imagery showing a wide angle showing the surrounding area in front of or near the distal end of its single-shaft, numerous imaging modules must be deployed on the distal end 103 of the single-shaft 101. However, due to the small dimensions of the distal end 103 of the single shaft 101, such an approach inevitably entails challenges associated with the difficulties the comes with the physical deployment of these numerous imaging modules. Also, non-continuous deployment of those image modules, which is typically the case, inevitably results in “blind areas/spots” surrounding the area in front of or near the distal end 103 of its single-shaft 101. Therefore, there is a need to address the aforementioned known disadvantages (e.g., the aforementioned difficulties and deficiencies) that usually comes with a conventional single-shaft endoscope.

### BRIEF SUMMARY

The present disclosure relates to an endoscope with self-encased and self-illuminating wide-angle panoramic imaging module at its distal end to capture images of a wide angle (which can be as wide as 360 degree or near 360 degree) about a lumen (such as a gastrointestinal lumen) where the distal end is interposed during an endoscopy session. The disclosed endoscope addresses the aforementioned known disadvantages that usually come with a conventional single-shaft endoscope. First, a single deployment of the disclosed wide-angle panoramic imaging module 200 captures a wide-angle view of the area in front of, or near, the distal end 103 of its single-shaft 101, thus rendering additional deployment of image modules either unnecessary or less imperative. Second, the wide-angle view captured by the disclosed wide-angle panoramic imaging module is usually continuous, thus addressing the “blind areas/spots” issues that usually inevitably comes with the conventional single-shaft endoscope which uses multi-camera deployment for capturing the same surrounding areas.

### BRIEF DESCRIPTION OF THE DRAWINGS

The description of the illustrative embodiments can be read in conjunction with the accompanying figures. It will be appreciated that for simplicity and clarity of illustration, elements illustrated in the figures, unless expressly specified, have not necessarily been drawn to scale. Also, any text and/or any numerical data (numbers) appeared on any drawing figures is provided to illustrate an exemplary embodiment or implementation, and thus is provided for the purpose of illustration and not for the purpose of limitation. For example, the dimensions of some of the elements may be exaggerated relative to other elements. Embodiments incorporating teachings of the present disclosure are shown and described with respect to the figures presented herein, in which:

FIG. 1 illustrates a conventional single-shaft endo scope in the related art.

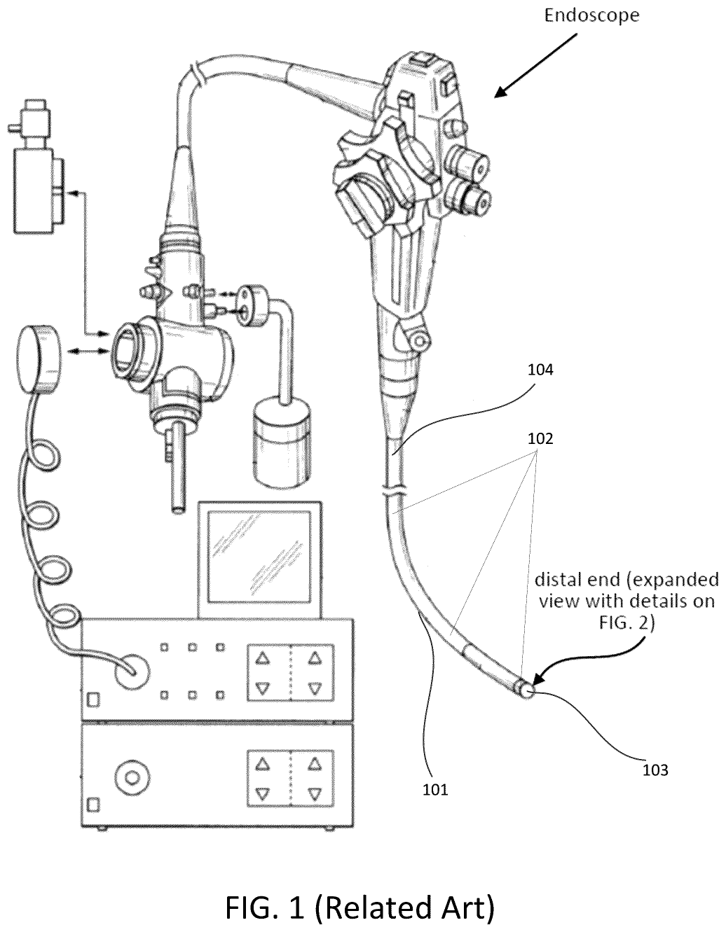

FIG. 2 illustrates the disclosed endoscope with self-encased and self-illuminating wide-angle panoramic imaging module 200 in accordance with one or more embodiments of the present disclosure.

### DETAILED DESCRIPTION

In the following detailed description of exemplary embodiments of the disclosure in this section, specific exemplary embodiments in which the disclosure may be practiced are described in sufficient detail to enable those skilled in the art to practice the disclosed embodiments. However, it is to be understood that the specific details presented need not be utilized to practice embodiments of the present disclosure. The following detailed description is, therefore, not to be taken in a limiting sense, and the scope of the present disclosure is defined by the appended claims and equivalents thereof.

References within the specification to “one embodiment,” “an embodiment,” “embodiments”, or “one or more embodiments” are intended to indicate that a particular feature, structure, or characteristic described in connection with the embodiment is included in at least one embodiment of the present disclosure. The appearance of such phrases in various places within the specification are not necessarily all referring to the same embodiment, nor are separate or alternative embodiments mutually exclusive of other embodiments. Further, various features are described which may be exhibited by some embodiments and not by others. Similarly, various requirements are described which may be requirements for some embodiments but not other embodiments.

The terminology used herein is for the purpose of describing particular embodiments only and is not intended to be limiting of the disclosure. As used herein, the singular forms “a”, “an” and “the” are intended to include the plural forms as well, unless the context clearly indicates otherwise. Moreover, the use of the terms first, second, etc. do not denote any order or importance, but rather the terms first, second, etc. are used to distinguish one element from another.

In the description, relative terms such as “left,” “right,” “vertical,” “horizontal,” “upper,” “lower,” “top” and “bottom” as well as any derivatives thereof (e.g., “left side,” “upper sub winding,” etc.) should be construed to refer to the logical orientation as then described or as shown in the drawing figure under discussion. These relative terms are for convenience of description and are not intended to convey any limitation with regard to a particular orientation.

Those of ordinary skill in the art will appreciate that the components and basic configuration depicted in the following figures may vary. Other similar or equivalent components may be used in addition to or in place of the components depicted. A depicted example is not meant to imply limitations with respect to the presently described one or more embodiments and/or the general disclosure.

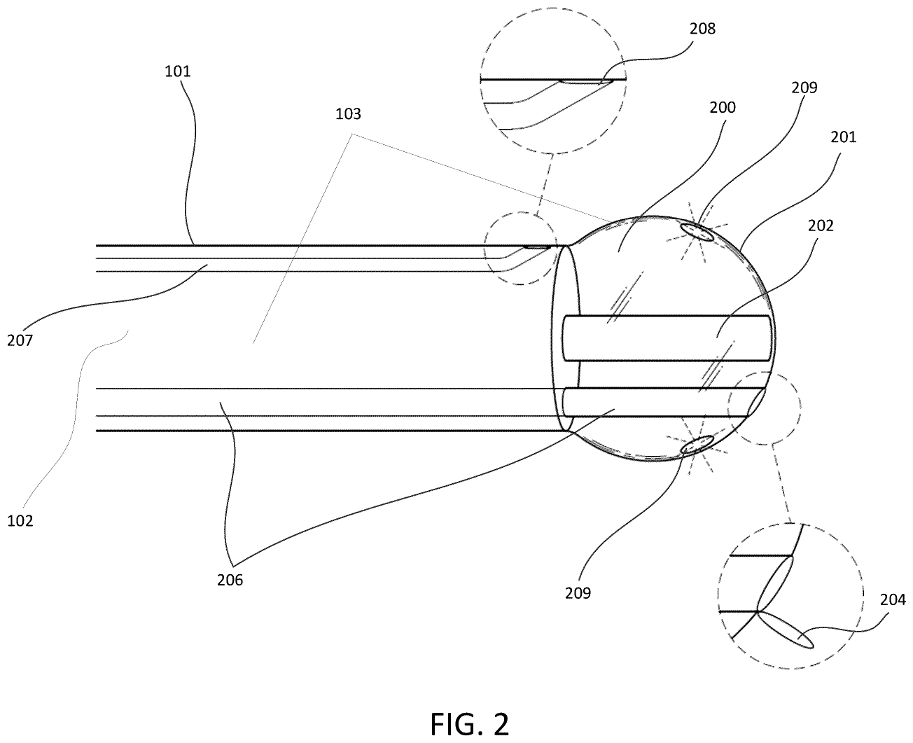

Referring to FIG. 2, in one embodiment, the dome-shaped housing (casing) 201 is provided at the distal end 103 of a single shaft 101 of an endoscope is optically clear (with respect to incoming and outgoing lights). The dome-shaped housing 201 is self-contained and self-encased (self-enclosed), and is self-sealed at least with respect to external water (water-proof).

In one embodiment, the camera housed inside the casing 201 is a single-lens wide-angle panoramic camera 202 capable of capturing a panoramic view of a wide angle, which may be as wide as 360 degrees or near 360-degrees. Such a panoramic camera is equipped with single-lens-based wide-angle panoramic imaging technologies already available and used in cameras designed to be used for various applications, such as sports-imaging (e.g., capturing surrounding real-time images during a sky-diving). One example of cameras using such single-lens-based wide-angle panoramic imaging technologies is 360Fly cameras, which is well-known among population fond of sports-imaging.

Referring to the same FIG. 2, in one embodiment, a flap 204 or gate 204 is provided on the self-encase housing 201. The flap 204 or gate 204 may be pushed to open, thus becoming an outlet for instrument channel 206 inside the hollow channel 102. A dome-washing channel 207 is provided inside the hollow channel 102, with its dome-washing nozzle 208 being positioned, as exemplified, to wash the casing 201.

Referring to the same FIG. 2, in one embodiment, the self-illumination may be achieved through one or more LEDs 209 provided in the interior of the self-encased housing.

As a skilled artisan appreciates, the disclosed endoscope with self-encased and self-illuminating wide-angle panoramic imaging module 200 addresses the aforementioned known disadvantages of a conventional endoscope. First, a single deployment of the disclosed wide-angle panoramic imaging module 200 captures a wide-angle view of the area in front of or near the distal end 103 of its single-shaft 101, thus rendering additional deployment of image modules either unnecessary or less imperative. Second, the wide-angle view captured by the disclosed wide-angle panoramic imaging module 200 is usually continuous, thus addressing the “blind areas/spots” issues that usually inevitably come with the conventional single-shaft endoscope which uses multi-camera deployment for capturing the same surrounding areas.

While the disclosure has been described with reference to one or more exemplary embodiments, it will be understood by those skilled in the art that various changes may be made and equivalents may be substituted for elements thereof without departing from the scope of the disclosure. In addition, many modifications may be made to adapt a particular system, device or component thereof to the teachings of the disclosure without departing from the essential scope thereof. Therefore, it is intended that the disclosure not be limited to the particular embodiments disclosed for carrying out this disclosure.

A person understanding the present disclosure may now conceive of alternative structures and embodiments or variations of the above all of which are intended to fall within the scope of the present disclosure as defined in the appended claims.

What is claimed is:

1. A single-shaft endoscope having a single shaft, comprising:
a housing at a distal end of the single shaft, the housing configured to be self-encased, optically clear, and self-illuminating; and
a camera deployed inside the housing, the camera configured to provide a wide-angle panoramic view.
